(19) United States
(12) Patent Application Publication
(10) Pub. No.: US 2003/0108980 A1
(43) Pub. Date: Jun. 12, 2003

(54) Bioluminescent methods for direct visual detection of environmental compounds

(76) Inventors: Gary S. Sayler, Blaine, TN (US); Steven A. Ripp, Knoxville, TN (US); John Sanseverino, Knoxville, TN (US)

Correspondence Address: BARBARA S. KITCHELL, AKERMAN, SENTERFITT & EIDSON, P.A., 222 Lakeview Avenue, Fourth Floor, P.O. Box 3188, West Palm Beach, FL, 33402-3188, US

(21) Appl. No.: 09/923,132

(22) Filed: Aug. 6, 2001

Related U.S. Application Data
(60) Provisional application No. 60/225,232, filed on Aug. 14, 2000.

(51) Int. Cl. C12Q 1/68; C12Q 1/04; C12N 1/21

(52) U.S. Cl. 435/34; 435/252.3

(57) ABSTRACT

The invention relates to devices and methods that utilize immobilized bacterial bioreporters genetically engineered to emit light visible to the naked eye in the presence of selected analytes. An exemplary bioreporter is an E. coli that has been modified to respond to mercury II as a result of incorporation of a merRop/lux gene cassette into its genome. Systems employing analagously engineered microorganisms can detect selected toxins quickly without need for expensive instruments or highly trained technicians.

This application claims priority as a continuation-in-part application based on U.S. provisional application Ser. No. 60/225,232 filed Aug. 14, 2000, the entire contents of which are herein incorporated by reference.

### 1.0 BACKGROUND OF THE INVENTION

1.1 Field of the Invention

The invention is concerned with detection methods and devices that utilize immobilized genetically engineered whole cells to detect selected chemical compounds. Methods and devices have been developed that are useful for rapid, direct visual detection of hazardous chemicals.

1.2 Description of the Related Art

Water supplies are particularly vulnerable to contamination by toxins and hazardous chemicals. There are instances where regional untested sources of water for personal use must be utilized; for example, in underdeveloped countries, in populated areas where water supplies have been compromised, or where natural disasters have made local drinking water supplies unsafe. In addition, many locales use well water as a primary source of drinking water that is not routinely tested; for example, rural farms and homes. Particularly at risk are military personnel who may find themselves in uncharacterized, hostile territories and must rely on local, untested sources of food and water for survival. Drinking water is usually obtained from untreated water sources such as ponds, streams, and wells and these sources may have dissolved waste materials as well as chemical toxins from both natural and agricultural sources.

Screening procedures for organic and inorganic constituents in water have traditionally employed well-known standardized methods. Naphthalene, toluene, phenol and mercury detection generally relies on expensive equipment such as gas chromatographs, high pressure liquid chromatography and atomic absorption and complex extraction procedures. These techniques require trained personnel and are therefore not routinely used where budgets and lack of skilled technicians must be taken into consideration.

Mercury is a particularly prevalent and persistent toxin that may be present in many environments. Long-term exposure to either organic or inorganic mercury can permanently damage the brain, kidneys, and developing fetuses. The form of mercury and the way people are exposed to it determine which of these health effects will be more severe. For example, organic mercury that is consumed in contaminated fish or grain may cause greater harm to the brain and developing fetuses than to the kidney; inhaled metallic mercury vapor may cause greater harm to the brain; and inorganic mercury salts that are eaten in contaminated food or consumed in water may cause greater harm to the kidneys. Maternal exposure to organic mercury may lead to brain damage in fetuses; while adults exposed to metallic mercury vapor may develop shakiness (tremors), memory loss, and kidney disease (ASTDR, 1990).

Short-term exposure to high levels of inorganic and organic mercury has similar health effects; but full recovery is more likely after short-term exposures, once the body clears itself of the contamination. Although mercury has not been shown to cause cancer in humans, the health effects resulting from short-term human exposure to inorganic mercury in water are not known. Based on animal studies, the estimated minimum risk level for inorganic mercury is approximately 0.8 parts per million.

In many situations, such as municipal water supplies, monitoring of contaminants is performed on an ongoing basis and acceptable levels of toxic chemicals can be controlled. Current detection and quantification methods are generally satisfactory to meet community standards; however, there in emergency situations such as natural disasters, there is a need for rapid means of determining acceptable levels of undesirable chemicals.

Detection of chemicals in water is therefore important in identifying potable water in undeveloped countries, in military operations, and in suspected instances of natural contamination. Simple qualitative dip-stick and litmus paper tests for common chemicals is desirable in order to provide real-time and inexpensive means to determine dangerous levels of toxic chemicals in water supplies.

### 2.0 SUMMARY OF THE INVENTION

The invention is particularly directed to developing methods of rapidly detecting chemical toxins without use of complicated detection systems by employing simple systems that provide near real-time results. By developing genetically modified bioluminescent bacteria as bioreporters, simple test-strip procedures have been developed that allow virtually immediate visual observation of a luminescent signal in the presence of a selected chemical inducer. Such a system has been developed for the visual detection of mercury compounds and is applicable to bioluminescent detection of other compounds such as naphthalene, phenol and related organic compounds.

Certain naturally occurring organisms harbor a gene that under appropriate conditions expresses a bioluminescent protein when an inducer compound is present. Examples include the jellyfish Aequorea victoria which expresses green fluorescent protein, firefly luciferase and bacteria such as Vibrio fischeri and P. fluorescens. For example, P. fluorescens will express a bioluminescent protein when induced by salicylate.

When the promoter for a wild-type gene expressing a luminescent protein is replaced with a promoter responsive to another compound, it is possible to engineer a cell that is selectively responsive to that compound. Using this approach, E. coli EC100 has been engineered to harbor a transcriptional fusion responsive to mercury compounds. This is illustrated in FIG. 1 where the merRo/p promoter is shown in a luxCDABE gene construct. FIG. 1 illustrates a cassette that can be incorporated into P. fluorescens or E. coli, making the bacterium responsive to mercury compounds.

The genetically modified bacteria respond to mercury II ion by producing a luminescence that is visible to the naked eye. The light is highly visible under night conditions and with the use of night goggles where protection from ambient light is provided.

In certain embodiments, bioreporter cells responsive to mercury II may be immobilized in a stabilizing matrix. Alginate has been successfully used for encapsulation of cells without adverse effects on viability. Long-term viability (weeks to months) is possible as long as the alginate-encased cells remain moist. Latex copolymers have also been reported to be useful for immobilizing E. coli and maintaining viability (Lyngberg, et al., 1999A). Other matrices include alginate, carrageenan, acrylic vinyl acetate copolymer, latex, polyvinyl chloride polymer, sol-gels, agar, agarose, micromachined nanoporous membranes, polydimethylsiloxane (PDMS), polyacrylamide, polyurethane/polycarbamyl sulfonate, polyvinyl alcohol and electrophoretic deposition.

Luminescent bioreporter bacteria may be immobilized on a variety of materials and still maintain the ability to respond to an inducer by producing bioluminescence. This has been demonstrated by immobilizing P. fluorescens 5R on cellulose strips using latex. In selecting a suitable immobilization material, one takes into consideration such factors as the access of the inducer to the bioreporter. If diffusion is slow or inhibited, there will be little or no response. Another factor is toxicity. Alternatively, the immobilization material may itself inhibit bioreporter response to the inducer and so be unsatisfactory as a support.

A number of other methods of immobilization may be useful, including incorporation of a cellulose-binding domain in the bioluminescent bioreporter bacteria. The cellulose-binding domain will physically bind the cells to a cellulose filter.

Cellulose-degrading bacteria produce an enzyme complex containing catalytic and non-catalytic domains. The function of the catalytic domain is to enzymatically degrade the cellulose into simple sugars. The function of the non-catalytic domain is to bind the enzyme complex to the insoluble cellulose. The non-catalytic region referred to as the cellulose-binding domain (CBD) has been identified in over 100 bacterial and fungal species and can be grouped into nine structural families (I-IX) based on amino acid homology. CBDs are peptides that range in length from 35 to >100 amino acid residues. Several gene sequences for these CBDs are available through GenBank.

In utilizing cellulose supports, the bioluminescent bioreporter may be genetically modified to express CBDs on the cell surface as a single protein or the CBDs may be fused to an existing cell surface protein. Particular configurations will be selected based on binding characteristics, e.g., strength of binding, to a selected substrate.

In certain embodiments, the invention comprises a filter strip on which an immobilized bioreporter bacterium is packaged in a sealable container suitable for long-term storage. The strips may be contacted with a target sample (e.g., water) and allowed to incubate. In the presence of the appropriate inducer, bioluminescence will be observed.

To illustrate response to visible light, E. coli EC100 was engineered to contain a merRo/p-lux fusion that emits light visible to the naked eye when exposed to mercury. When immobilized on cellulose strips, E. coli EC100 emitted visible light less than one hour after test strips were contacted with a solution containing the inducer salicylate. The light was readily observed with the naked eye, night vision equipment, or other portable device that allows direct observation, such as through use of a light-tight slide holder.

In a particular aspect of the invention, methods have been developed for use of handheld devices for the rapid detection of divalent mercury. The device comprises a genetically engineered reporter microorganism that bioluminesces in the presence of aqueous-phase divalent mercury (Hg²⁺). The device has the following features:

1. Capable of detecting Hg²⁺ at approximately 1 part per million;

2. The assay is rapid, with a response time of approximately 30 minutes;

3. The assay results are unambiguous to the field personnel;

4. Positive and negative controls can be included;

5. Is easily portable;

6. Bioluminescence is observable with the human eye under daylight or night conditions; and,

7. Maintains a shelf life at room temperature.

Kits are also contemplated as part of the invention. Immobilized bioluminescent reporter microorganisms such as P. fluorescens 5RL as described may be conveniently packaged individually or in bulk so as to be conveniently stored, transported and dispensed. For example, impregnated filter strips may be packaged in factory-sealed aluminum packaging convenient for single sample use. Each package will contain the bacteria immobilized filter strip and a medium, liquid or semi-solid, for preservation of the culture, allowing for long-term storage. The size of the packets may be from about 3.5 in long and about 1.5 in wide, or of a dimension small enough for carrying on the person such as in a pocket or packed in a field bag. The kits will also typically contain directions for use and, optionally, contain light-tight holders and/or night goggles for light detection.

Accordingly, devices for detecting selected analytes are one embodiment of the invention. An exemplary device will include a stably transformed microorganism, preferably a bacterium, that harbors a promoterless gene cassette incorporating a regulatory element responsive to a selected analyte. The gene cassette, preferably a lux gene cassette, will be incorporated into the bacterial genome. In the case of the lux gene, the cassette may be merRo/p-lux where the lux gene comprises CDABE. The mer operator determines that the system will respond to mercury II; however, other regulatory elements could be selected that are responsive to other chemicals, such as naphthalene, toluene, dichlorophenoxyacetic acid and the like. While the invention has been illustrated with E. coli and P. fluorescens, other bacteria can be used. Some examples, not to be considered limiting, are found in Table 4.

An advantage of the disclosed device is that the bioreporters can be immobilized onto supports such as cellulose in a filter paper strip configuration. Generally one will desire to immobilize in a substance compatible with bacterial survival, such as the matrices discussed herein. A preferred embodiment is a latex immobilization material. The immobilized bioreporter may then be supported on a material such as cellulose. Once immobilized in this manner, the bioreporters are easily packed and transported for use by individuals without additional equipment.

The device may be modified for easy handling such as an apparatus comprising the device in a simple holder that is readily hand manipulated. A battery operated apparatus is also envisioned where test strips are automatically popped out of a container, dipped into water to be tested, then automatically popped back into the container with a simple push button. The container can include a moist environment and be supplemented with media. Numerous modifications of this basic scheme are envisioned and would be apparent to one skilled in the art.

An important aspect of the invention is the genetically modified microorganism employed as a bioreporter for a selected compound. In a preferred embodiment, a genetically modified bacterium is constructed that responds by producing luminescence in the presence of mercury II. A list of other compounds for which promoters have been identified could be used in a similar manner to construct selective bioreporters. It is believed that preferred constructs can be prepared by selecting “high producers” in terms of selecting the light producing genes of microorganisms, selecting an appropriate promoter, and engineering a bacterium or other appropriate microorganism so that the construct is chromosomally integrated into the bioreporter cell.

### 3.0 BRIEF DESCRIPTION OF THE DRAWINGS

FIG. 1 is a schematic showing the basic construction of a bioreporter cell. Upon exposure to a

specific analyte, the promoter/reporter gene complex is transcribed into messenger RNA (mRNA) and then translated into a reporter protein that is ultimately responsible for generating a signal.

FIG. 2 shows bioluminescence emitted from individual colonies of microbial cells that harbor genes for bacterial luciferase.

FIG. 3 shows a schematic of plasmid pFSD3. The merRo/p fragment was blunt end cloned into the BamHI-SmaI site upstream of the lux gene cassette. P represents PshAI and ME=mosaic ends.

FIG. 4A and FIG. 4B represent growth curves of E. coli EC100, ARL1, ARL2 and ARL3. FIG. 4A shows optical density changes with time for the merRo/p-lux bioreporter strains ARL1, ARL2 and ARL3 and wild-type E. coli EC100. FIG. 4B shows a linear regression of the linear portion of the data. The slopes of these lines represent the growth rate of each strain. From the data, the doubling times for E. coli EC100, ARL1, ARL2 and ARL3 are 1.61, 1.61 and 1.67 and 1.58 hours respectively.

FIG. 5 shows the time course of bioluminescence production after exposure to 0.5 ppm HgCl₂ added at time zero for strains E. coli ARL1, ARL2 and ARL3.

FIG. 6A and FIG. 6B is a comparison of salicylate-induced P. fluorescens 5RL cells with and without latex. FIG. 6A shows immobilized cells on a nylon membrane. FIG. 6B shows the bioluminescence production of the immobilized cells.

FIG. 7 shows induction of P. fluorescens 5RL by salicylate in the presence and absence of Rovace SF-091.

FIG. 8 shows a schematic representation of a test strip.

FIG. 9A and FIG. 9B shows results of bioreporter test strips tested against various chemicals. FIG. 9A is a test strip with 6 wells of P. flurorescens 5RL on the left. On the right are three bioreporters, P. fluorescens 5RL, P. putida TVA8 and E. coli ARL1 with the positive control P. putida AL2060. FIG. 9B is a dark room image of each strip. The strip on the right was exposed to a mixture of toluene, naphthalene and mercury. The strip on the left was exposed to salicylate.

FIG. 10 shows bioluminescence response for immobilized in a simulation of test strip performance. Three bioreporters, P. fluorescens 5RL, P. putida TVA8 and E. coli ARL1 with the positive control P. putida AL2060 were exposed to a mixture of toluene, naphthalene and mercury.

FIG. 11 shows an alternate test strip design. This design employs lyophilized cells to extend the shelf life of the test strip. This design simplified the test strip by elimination of any water barriers and an alternate method for applying the lyophilized cells to the strip.

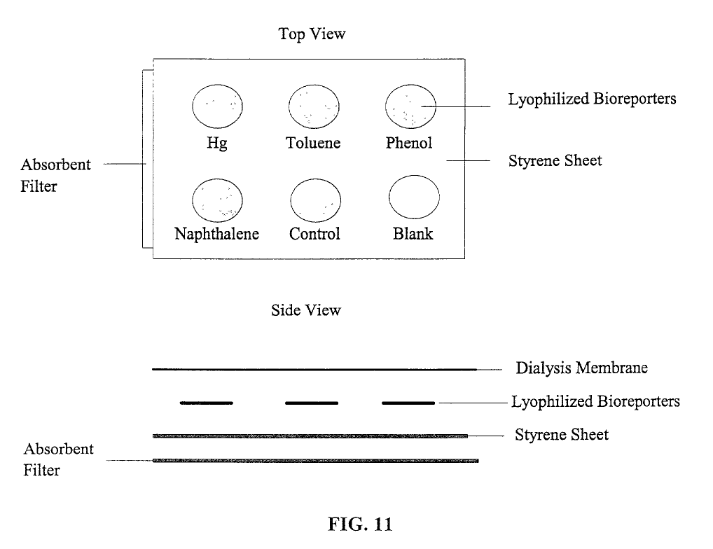

FIG. 12 shows a schematic representation of pFSD3 containing the merRo/pA fused with the lux genes.

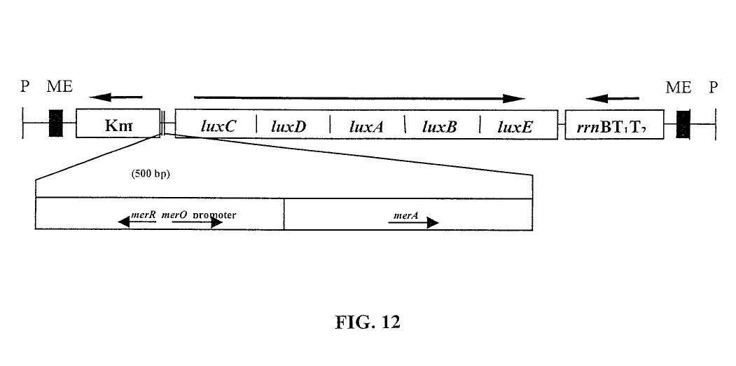

### 4.0 DETAILED DESCRIPTION

The present invention discloses methods and bioreporter systems that take advantage of a class of whole cell bioluminescent bioreporters. Bacterial bioreporters in particular have recently been of interest and have focused attention on the luminescent products that are produced in several types of organisms.

Bacteria constantly interrogate their surrounding environment in search of utilizable substrates for carbon and energy and potential toxins. Specific bacteria and associated genetic systems have been isolated and identified that elicit a response in the presence of naphthalene (Sanseverino et al., 1993), toluene (Gibson et al., 1966), phenol (Folsom et al., 1990) and mercury (Summers, 1986). By combining specific genetic responsive elements with bioluminescence, bioreporter strains are attractive candidates for using in inexpensive methods to screen for specific pollutants. Typically the light produced when the lux gene product is expressed is detected by means such as a photo counter-imaging device, a scintillation counter, a luminometer or photographic film.

### 4.1 Background of Bacterial Bioreporters

### 4.1.1 Bacterial Bioreporters

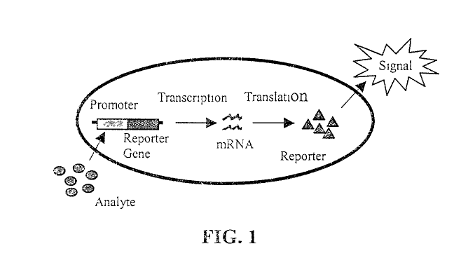

Bioreporters refer to intact, living microbial cells that have been genetically engineered to produce a measurable signal in response to a specific chemical or physical agent in their environment. Bioreporters contain two essential genetic elements, a promoter gene and a reporter gene. The promoter gene is turned on (transcribed) when the target agent is present in the cell's environment. The promoter gene in a normal bacterial cell is linked to other genes that are then likewise transcribed and translated into proteins that allow the cell to either combat or adapt to the agent to which it has been exposed. In the case of a bioreporter, these genes, or portions thereof, have been removed and replaced with a reporter gene. Consequently, turning on the promoter gene now causes the reporter gene to be turned on. Activation of the reporter gene leads to production of reporter proteins that ultimately generate some type of a detectable signal. This process is schematically represented in FIG. 1. Therefore, the presence of a signal indicates that the bioreporter has sensed a particular target agent in its environment.

Originally developed for fundamental analysis of factors affecting gene expression, bioreporters have been employed for the detection of environmental contaminants (King et al., 1990) and have since evolved into fields as diverse as medical diagnostics, precision agriculture, food-safety assurance, process monitoring and control, and bio-microelectronic computing. Their versatility stems from the fact that there exist a large number of reporter gene systems that are capable of generating a variety of signals. Additionally, reporter genes can be genetically inserted into bacterial, yeast, plant, and mammalian cells, thereby providing considerable functionality over a wide range of host vectors.

### 4.1.2 Reporter Gene Systems

Several types of reporter genes are available for use in the construction of bioreporter organisms, and the signals they generate can usually be categorized as either colorimetric, fluorescent, luminescent, chemiluminescent or electrochemical. Although each functions differently, their end product always remains the same—a measurable signal that is proportional to the concentration of the unique chemical or physical agent to which they have been exposed. In some instances, the signal only occurs when a secondary substrate is added to the bioassay (luxAB, Luc, and aequorin). For other bioreporters, the signal must be activated by an external light source (GFP and UMT), and for a select few bioreporters, the signal is completely self-induced, with no exogenous substrate or external activation being required (lucCDABE). The following sections outline in brief some of the reporter gene systems available and their existing applications.

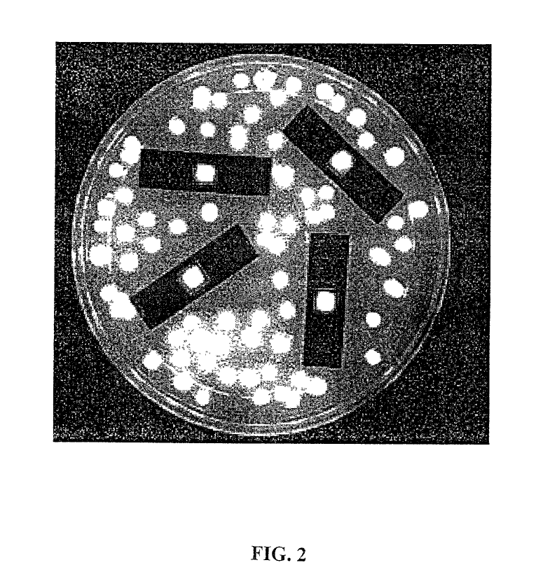

Bacterial luciferase (Lux): Luciferase is a generic name for an enzyme that catalyzes a light-emitting reaction. Luciferases can be found in bacteria, algae, fungi, jellyfish, insects, shrimp, and squid, and the resulting light that these organisms produce is termed bioluminescence. In bacteria, the genes responsible for the light-emitting reaction (the lux genes) have been isolated and used extensively in the construction of bioreporters that emit a blue-green light with a maximum intensity at 490 nm (FIG. 2) (Meighan, 1994). Three variants of lux are available, one that functions at <30° C., another at <37° C., and a third at <45° C. The lux genetic system consists of five genes, luxA, luxB, luxC, luxD, and luxE. Depending on the combination of these genes used, several different types of bioluminescent bioreporters can be constructed.

luxAB bioreporters: luxAB bioreporters contain only the luxA and luxB genes, which together are responsible for generating the light signal. However, to fully complete the light-emitting reaction, a substrate must be supplied to the cell. Typically, this occurs through the addition of the chemical decanal at some point during the bioassay procedure. Numerous luxAB bioreporters have been constructed within bacterial, yeast, insect, nematode, plant, and mammalian cell systems.

luxCDABE bioreporters: Instead of containing only the luxA and luxB genes, bioreporters can contain all five genes of the lux cassette, thereby allowing for a completely independent light generating system that requires no extraneous additions of substrate nor any excitation by an external light source. So in this bioassay, the bioreporter is simply exposed to a target analyte and a quantitative increase in bioluminescence results, often within less than one hour. Due to their rapidity and ease of use, along with the ability to perform the bioassay repetitively in real-time and on-line, makes luxCDABE bioreporters extremely attractive. Consequently, they have been incorporated into a diverse array of detection methodologies ranging from the sensing of environmental contaminants to the real-time monitoring of pathogen infections in living mice. FIG. 2 shows bioluminescence emitted from individual colonies of microbial cells containing the genes for bacterial luciferase.

Nonspecific lux bioreporters: Nonspecific lux bioreporters are typically used for the detection of chemical toxins. They are usually designed to continuously bioluminesce. Upon exposure to a chemical toxin, either the cell dies or its metabolic activity is retarded, leading to a decrease in bioluminescent light levels. Their most familiar application is in the Microtox® assay where, following a short exposure to several concentrations of the sample, the decreased bioluminescence can be correlated to relative levels of toxicity (Hermans et al., 1985).

Firefly luciferase (Luc): Firefly luciferase catalyzes a reaction that produces visible light in the 550-575 nm range. A click-beetle luciferase is also available that produces light at a peak closer to 595 nm. Both luciferases require the addition of an exogenous substrate (luciferin) for the light reaction to occur. Numerous luc-based bioreporters have been constructed for the detection of a wide array of inorganic and organic compounds of environmental concern. Their most promising application, however, probably lies within the field of medical diagnostics. Insertion of the luc genes into a human cervical carcinoma cell line (HeLa) illustrated that tumor-cell clearance could be visualized within a living mouse by simply scanning with a charge-in coupled device camera, allowing for chemotherapy treatment to rapidly be monitored on-line and in real-time (Contag et al., 2000). In another example, the luc genes were inserted into human breast cancer cell lines to develop a bioassay for the detection and measurement of substances with potential estrogenic and antiestrogenic activity (Legler et al., 1999).

Aequorin: Aequorin is a photoprotein isolated from the bioluminescent jellyfish Aequorea victoria. Upon addition of calcium ions (Ca²⁺) and coelenterazine, a reaction occurs whose end result is the generation of blue light in the 460-470 nm range. Aequorin has been incorporated into human B cell lines for the detection of pathogenic bacteria and viruses in what is referred to as the CANARY assay (Cellular Analysis and Notification of Antigen Risks and Yields) (Rider et al., 1999). The B cells are genetically engineered to produce aequorin. Upon exposure to antigens of different pathogens, the recombinant B cells emit light as a result of activation of an intracellular signaling cascade that releases calcium ions inside the cell.

Green fluorescent protein (GFP): Green fluorescent protein (GFP) is also a photoprotein isolated and cloned from the jellyfish Aequorea Victoria (Misteli and Spector, (1997). Variants have also been isolated from the sea pansy Renilla reniformis. GFP, like aequorin, produces a blue fluorescent signal, but without the required addition of an exogenous substrate. All that is required is an ultraviolet light source to activate the fluorescent properties of the photoprotein. This ability to autofluoresce makes GFP highly desirable in biosensing assays since it can be used on-line and in real-time to monitor intact, living cells. Additionally, the ability to alter GFP to produce light emissions besides blue (i.e., cyan, red, and yellow) allows it to be used as a multianalyte detector. Consequently, GFP has been used extensively in bioreporter constructs within bacterial, yeast, nematode, plant, and mammalian hosts. The use of GFP has revolutionized much of what we understand about the dynamics of cytoplasmic, cytoskeletal, and organellar proteins and their intracellular interactions.

Uroporphyrinogen (Urogen) III Methyltransferase (UMT): UMT catalyzes a reaction that yields two fluorescent products which produce a red-orange fluorescence in the 590-770 nm range when illuminated with ultraviolet light (Sattler et al., 1995). So as with GFP, no addition of exogenous substrates is required. UMT has been used as a bioreporter for the selection of recombinant plasmids, as a marker for gene transcription in bacterial, yeast, and mammalian cells, and for the detection of toxic salts such as arsenite and antimonite.

β-Galactosidase. β-galactosidase, encoded by the lacZ gene, is a key enzyme in the metabolism of lactose. This enzyme cleaves lactose to glucose and galactose. Several chromogenic substrates that are cleaved by β-galactosidase have been developed. β-galactosidase transforms the chromogenic substrate o-nitrophenol-β-D-galactopyranoside (ONPG) to a yellow product (405 nm), chlorophenol red-β-D-galactopyranoside (CPRG) to a red product (540 nm) and 5-bromo-4-chloro-3-indolyl-β-D-galactoside to a blue product (620 nm).

### 4.1.3 Bioluminescent Bioreporters for Mercury

Three mer-lux (HgII) plasmid biosensors (pRB28, pOS14, and pOS15) have been described based on bacterial luciferase genes (Selifonova et al, 1993). Plasmid pRB28 contains the merRo/pT′ fused to the luxCDABE operon of Vibrio fischeri. This mer fragment encodes the regulatory protein and operator/promoter for the mer operon. Mercury (Hg²⁺) enters the cell by passive diffusion. Plasmids pOS14 and pOS15, in addition to the merRo/pT′ contain the genes for active transport of Hg²⁺ and the complete mer operon, respectively. The host organism was Escherichia coli HMS 174. The highest sensitivities were achieved in minimal medium and were 1, 0.5, and 25 nM Hg²⁺, respectively (Selifonova et al, 1993). The drawback of this system is the plasmid nature of the bioreporter. Plasmids require constant antibiotic selection otherwise the host organism will not retain the plasmid.

Virta et al., (1995) and Petanen et al., (2001) have developed mercury biosensors based on the firefly luciferase from Photinus pyralis. While the detection limit for these strains was in the low nM range, the light reaction requires the addition of click beetle luciferin as a substrate for the light reaction. This requirement makes these constructs unsuitable for rapid, remote screening of water samples.

### 4.2 Direct Visualization of Luminescence in Presence of Inducers

The present invention has developed bioreporter systems that will allow near real-time visualization of a luminescent signal in the presence of an inducer compound. Light-emitting bacteria have been modified to express a light-emitting protein in the presence of selected inducer compounds. An exemplary bacterial construct, E. coli, was genetically engineered to contain a merRo/p-lux fusion that expresses a mercuric regulatory protein and operator/promoter for the mer operon. In the presence of mercury, the bacteria emit light visible to the naked eye. The modified bacteria have been immobilized on a suitable substrate and may be incorporated into a convenient hand-held device that is readily adapted for field use.

### 4.3 Construction of Mer-Lux Whole Cell Bioluminescent Reporters

Two bioluminescent organisms that respond to divalent mercury were constructed. Relevant features are listed in Table 1. The first strain was a merRo/p-lux construct. This strain has the regulatory elements of the mer operon. The second strain contains a merRo/pA-lux fusion. The strain contains the mercuric reductase (merA) which reduces Hg²⁺ to Hg⁰. In cases of high mercury contamination, the presence of merA mitigates mercury toxicity so that a light response will still be observed.

TABLE 1
Strains and plasmids

| Plasmid | Relevant Features | References |
|---|---|---|
| PUTK2 | Control lux plasmid | Layton et al., 1998 |
| pFSP-3 | PMOD-2, Km^{r}, luxCDABE, | |
| | T₁T₂ termination sequence | |
| pUT-merRo/p-lux | Km^{r}, mer regulatory region | |
| | and operator | |
| pUT-merRo/pA-lux | Km^{r}, mer regulatory region | |
| | and operator, mercuric | |
| | reductase | |

Applegate et al., 1998 reported use of a transposable element to integrate a lux reporter into bacteria. The basic approach consists of inserting a regulatory element of interest in front of the promoterless luxCDABE gene cassette in a modified MiniTn5 transposon. The transposon is provided in trans on a delivery vector such as pUT (De Lorenzo et al, 1993) resulting in stable chromosomal insertions. This system was used to construct whole cell bioluminescent reporters for the chemical compounds indicated in Table 2.

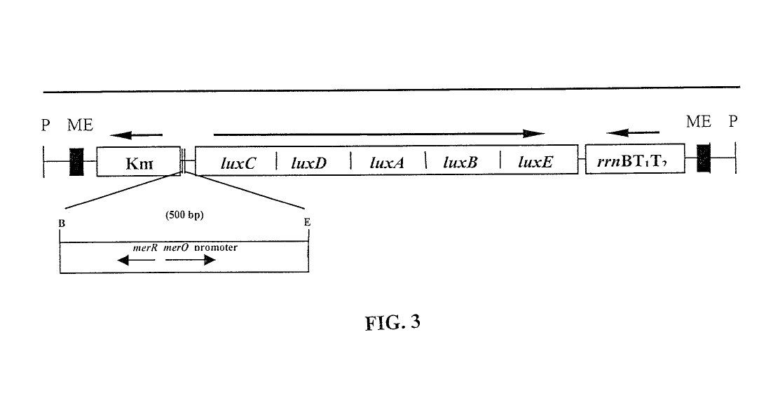

Modification of this approach is shown in FIG. 3 showing a schematic of the modified transposon vector based on the pMOD EZ:TN^{tm} system of Epicenter Technologies (Madison, Wis.). The incorporation of the Photorhabdus luminescens lux CDABE genes, the kanamycin resistance gene and the T₁T₂ termination sequences illustrate the modifications.

TABLE 2
Chromosomally-encoded Whole Cell bioluminescent Reporters

| Bioluminescent Reporter | lux Fusion | Reference |
|---|---|---|
| Pseudomonas fluorescens | nahRG-lux fusion for | King et al., 1990 |
| HK44 | the detection of | |
| | naphthalene | |
| Pseudomonas putida | Chromosomal-based | Applegate et al., 1998 |
| TVA8 | tod-lux fusion for the | |
| | detection of toluene | |
| Ralstonia euthropha | tfd-lux for the | Hay et al., 2000 |
| JMP134-32 | detection of the | |
| | herbicide 2,4-D | |
| Ralstonia eutropha | bph-lux for the | Layton et al., 1998 |
| ENV307 | detection of | |
| | polychlorinated | |
| | biphenyls | |

### 5.0 EXAMPLES

### 5.1 Example 1

### Construction of Chromosomally-Based mer-lux Whole Cell Bioluminescent Reporters

Three bioluminescent reporter strains containing the merR-lux construct incorporated in the E. coli chromosome were constructed as described. A schematic of the pFSP-3 vector used in these constructs including incorporation of the luxCDABE genes, the kanamycin resistance gene, and the termination sequences is shown in FIG. 3. The vector was a gift from Dr. Bruce M. Applegate, (Purdue University, West LayFayette, Ind.).

A 505 bp merR fragment was previously PCR amplified from the mer operon and cloned into the TA Cloning Vector (pCR2.1; Invitrogen, San Diego, Calif.). Primers (sequence ID 1 and 2; Table 3) for the amplification were synthesized based on the merRo/p sequence listed in GenBank (Accession #AF071413; nucleotides 19133-19638). The source of the mer DNA was pDG106 (Gambill and Summers, 1985). The merR was excised from pCR2.1-merR with EcoRV and BamHI. Plasmid pFSP3 was prepared by digesting with SmaI and BamHI, dephosphorylation by shrimp alkaline phosphotase (USB, city, State) and purification by GeneClean (company address). The merR fragment was ligated into pFSP3 overnight at 16° C. followed by chemical transformation into chemically competent E. coli DH5α cells. Transformants were subjected to miniprep plasmid isolation and father screened by restriction digestion with BamHI and KpnI. A positive clone designated #7 contained the merR gene in the proper orientation to induce bioluminescence in the presence of Hg²⁺ ions. Following large scale preparation, the transposon vector was digested with PshAI overnight at 25° C. The 8.5 kb fragment containing the mer-lux reporter transposon was gel purified with Gene Clean. The transposome was formed by incubating the 8.5 kb fragment (mer lux EZ::TN) with transposase according to manufacturer's directions. The resultant transposome was then electroporated into E. coli EC 100 competent cells (Epicenter, Madison, Wis.). Electroporants were plated on LB agar plates with Km (50 mg/L). Three colonies were recovered which produced bioluminescence in the presence of Hg²⁺ ions. These strains were designated E. coli ARL1, E. coli ARL2, and E. coli ARL3.

The merR-lux works by passive diffusion of Hg²⁺ into the cell. It is advantageous to clone the merRTPC cluster into the transposon because this fragment is responsible for active transport of mercury into the cell. Accordingly, it is believed that at least one advantage of inserting this fragment is a more rapid uptake and visualization of bioluminescence. The merRTPC fragment (˜1.7 kb in length) is relatively large and may require some modification for cloning into pFSP3. Parameters that may be adjusted to achieve insertion include vector to insert ratio, ligation time, and ligation temperature.

### 5.2 Example 2

### Growth of Strains ARL1, ARL2, and ARL3

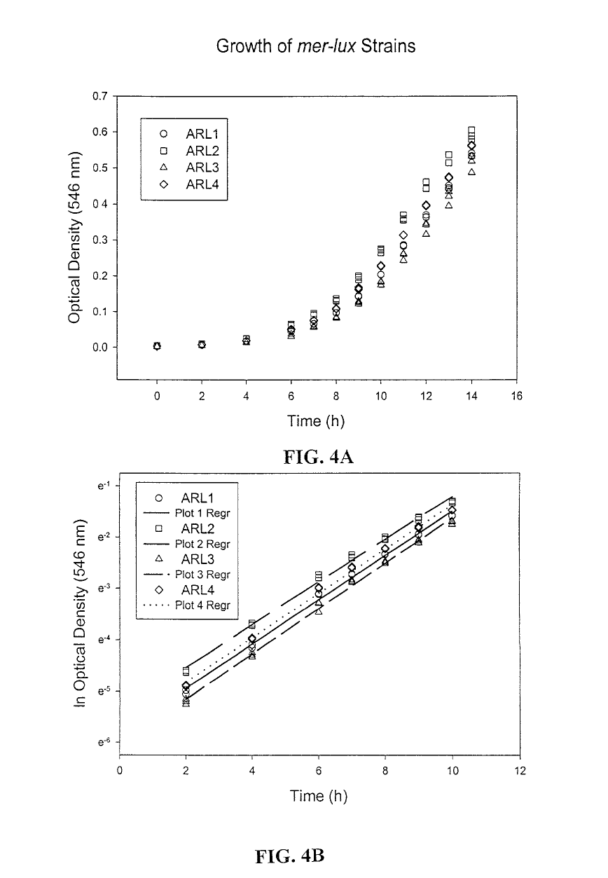

The growth rate of the bioluminescent E. coli strains was tested to determine if the mer-lux transposon was incorporated into a critical pathway in E. coli EC 100. Each transposon mutagenized strain, including the unmutagenized host strain, was grown in MSM broth supplemented with glucose (1 g/L), thiamine (1.0 mg/L), isoleucine (100 mg/L) and leucine (100 mg/L). The experiment was performed at 37° C. with shaking. Each growth curve was performed in triplicate. Only minor differences in growth rates were observed (FIGS. 4A and 4B). From the data in FIG. 4B, the doubling time of each strain can be determined by the following equation: T_{d}=ln 2 divided by the slope of the regression line. From the data, the doubling time for E. coli EC100, ARL1, ARL2 and ARL3 are 1.61, 1.61, 1.67 and 1.58, respectively. These results indicated that there are no significant mutations affecting critical growth pathways in each strain.

### 5.3 Example 3

### Detection of Mercury Using Bioluminescence

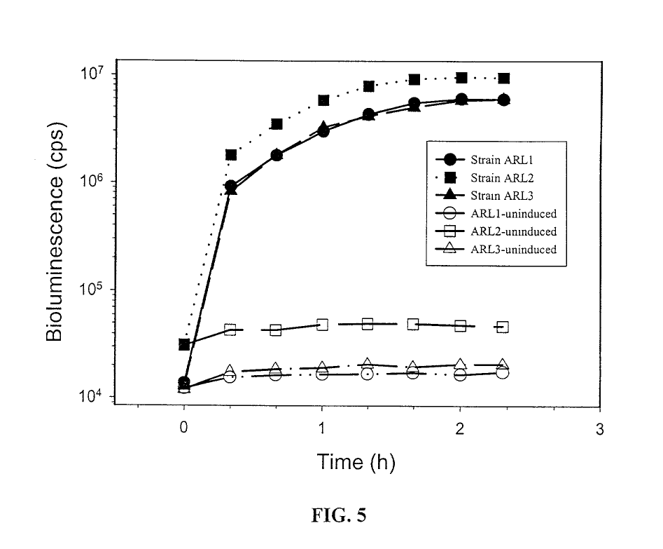

Strains ARL1, ARL2, and ARL3 were screened for bioluminescence production in the presence of HgCl₂. Each strain was grown in LB medium to an OD₅₄₆ₙₘ of 0.35 at which time 500 μg HgCl₂/L was added. Induction of bioluminescence in the presence of mercury was rapid (FIG. 5) with significant light produced in 30 minutes. Strain ARL2 had higher background bioluminescence (˜31,486 cps) in the absence of mercury relative to strain ARL1 and ARL3 (˜13,798 and ˜12,762 respectively). After 20 minutes, strain ARL2 produced approximately twice the amount of bioluminescence ((˜1,791,560 cps) relative to strains ARL1 and ARL3 (˜917.496 and 825,240 cps respectively).

### 5.4 Example 4

### Effect of Immobilization on Bioluminescent Cells

Many immobilization materials for cells are known and available; however, for practical applications where rapid development of visible luminescence is important, the effect of the immobilization material on cell growth and production of bioluminescence as well as toxicity on the cell must be determined.

### 5.5 Example 5

### Toxicity

Three latex mixtures were prepared from Rovace SF-091, Rhoplex SF-012, and Rhoplex-3122 (Rolm and Haas, Philadelphia, Pa.) were compared to determine toxicity effects on Pseudomonas fluorescens 5RL. Uninduced, log-phase cells (OD₅₄₆ₙₘ=0.35) were washed with mineral salts buffer (MSM) and resuspended in MSM+15% glycerol. Cells were spotted onto a cellulose membrane and excess moisture was allowed to dry. The cell spots were coated with each latex mixture at full strength and dried overnight at 5° C. and at 40% humidity.

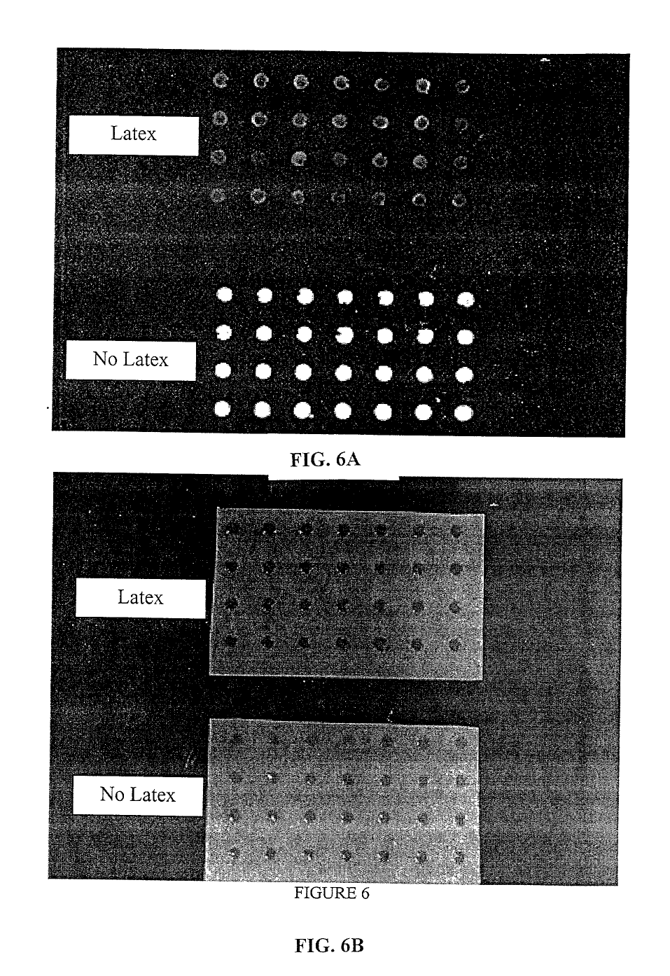

Viability of cells was determined by laying the membranes on LB broth supplemented with sodium salicylate (100 ppm) and monitoring light production. Cells coated with Rhoplex SF-012 and Rhoplex-3 122 produced no bioluminescence after exposure to salicylate. Rovace SF-091 supported the production of bioluminescence and maintained viability. Bioluminescence production was slightly diminished as compared to the non-latex control (FIGS. 6A and 6B).

FIGS. 6A and 6B show a comparison of salicylate-induced P. fluorescens 5RL cells with and without latex. FIG. 6A shows immobilized cells on a nylon membrane. FIG. 6B shows the immobilized cells.

### 5.6 Example 6

### Cell Growth

A growing cell assay experiment was performed in a microtiter plate using P. fluorescens 5RL in the presence and absence of Rovace SF-091. The purpose of the experiment was to demonstrate the inhibitory effect of the latex on the bioluminescent response of the cells. The cells were immobilized on a nylon membrane (BIOTRANS Nylon Membranes, ICN Biomedical, Aurora, Ohio) with a pore size of 0.45 μm. The membrane was used with a dot blot filter apparatus (Bio-Rad, Hercules, Calif.). Cells were place in individual wells in the dot-blot apparatus and transferred to the membrane by applying a vacuum. The number of cells per patch was calculated to be 1×10⁷ CFU. The vacuum was maintained until the cell patches appeared dry. One half of the membrane was placed on a slanted surface and the latex was poured over the membrane until it was covered. Excess latex drips off the membrane leaving a thin latex film over the membrane. The latex was dried at 5° C. and at 40% humidity overnight. The other half of the membrane was stored overnight at the same temperature and humidity. The next day, the individual patches were cut and placed on top of a sponge (3M ScotchBrite) slightly larger than the patches. The sponges were autoclaved and lyophilized prior to use. The sponge pieces and patches were placed in the wells of a 24 well microtiter plate. LB medium supplemented with sodium salicylate at a concentration of 100 ppm and tetracycline was added to each well. The plate was placed in the Wallac Luminometer (Gaithersburg, Md.) and bioluminescence readings were recorded every 30 minutes for 7 hours.

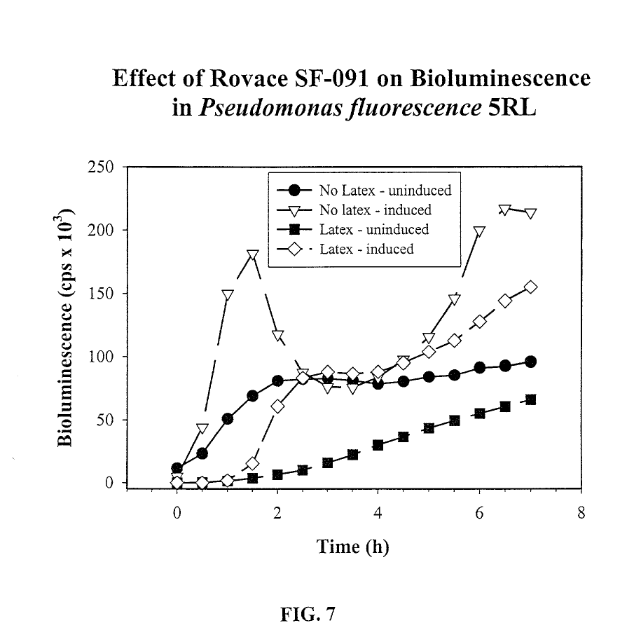

In the absence of salicylate, strain 5RL exhibited its typical response with a sharp increase in light production in the first 90 minutes followed by a gradual reduction during 90-240 minutes followed by another increase in light production (FIG. 7). In the presence of latex, bioluminescence was not observed until t=90 minutes followed by a plateau at 180-240 minutes. After 240 minutes, there was a steady increase in bioluminescence (FIG. 7). The lag phase may be due to diffusion of salicylate through the latex matrix or physiological effect on the cells themselves.

### 5.7 Example 7

### Test Strip Design

Design of a test strip takes several factors into consideration including physical layout, encapsulation of cells, delivery of nutrients, uptake of test water, and shelf life. Several variations of test strip construction are possible of which some are disclosed herein.

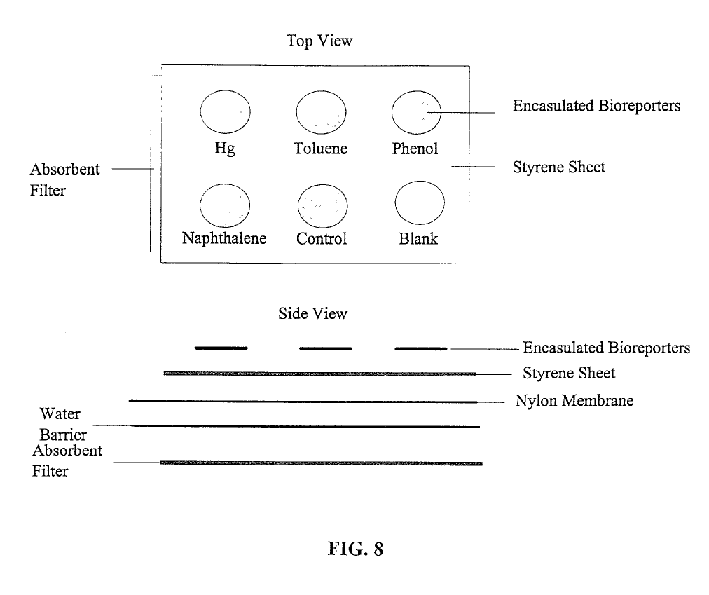

In this example, the test strip is the size of a credit card (84×54 mm) and has five layered parts (FIG. 8), each described as follows:

1. The bottom layer is absorbent Whatman filter paper. The dimensions of this filter paper are 89×54 mm. The filter paper is prepared by steam sterilization for 20 minutes followed by a 10 minute drying cycle. The sterile filter paper is soaked in YEPG broth (yeast extract (0.2 g/L)—peptone (2.0 g/L)—glucose (1.0 g/L)—NH₄NO₃ (0.2 g/L)). After the filter is saturated, the excess broth is decanted, the filter paper frozen at −80° C., and lyophilized. The result is an absorbent filter paper impregnated with freeze-dried YEPG. This will serve as a nutrient source for the bioreporters when they are ‘rehydrated’ with a contaminated water sample.

2. The second layer is an impermeable water barrier. Currently we are using aluminum foil. The purpose is to separate the YEPG-impregnated filter paper from the cells during storage.

3. The third layer is a nylon membrane (0.45 μm). This layer is secured to the fourth layer (styrene sheet) using a spray adhesive (3M Super77 Multi-Purpose Spray Adhesive). The purpose of this layer is to support the encapsulated cells.

4. The fourth layer is the styrene sheet. It acts as a semi-rigid support for the device as well as a mold for the encapsulated cells. Currently we are using styrene that is 0.5-1.0 mm thick. One-half inch circles are excised from the sheet (FIG. 6, top view). There is one circle for each bioreporter.

5. The last layer is the encapsulated cells. The current procedure is to grow the cells overnight in minimal salts medium (MSM) supplemented with trace elements and glucose. Cells are harvested by centrifugation at 5,000 rpm for 15 minutes. Cells are washed three times in either nitrogen-free MSM or phosphate-buffered saline. The cells are mixed with 1% noble agar, supplemented with trace elements and glucose, at a 1:1 ratio. This mixture is poured into the ½ inch diameter styrene mold and the agar/cell mixture is allowed to solidify.

6. All the pieces are assembled using acetate sealing tape.

7. The device is packaged in an air-tight, water tight material. Exemplary packaging may be either a heat-sealable foil or heat-sealable plastic (such as a “Seal-a-Meal” system).

The user removes the device from its package and removes the aluminum foil impermeable barrier. The device can be dipped into the test water with the extended absorbent wick face down. Water will be wicked up the filter paper. The test water-saturated filter paper will dissolve the freeze-dried YEPG. The contaminants and the YEPG nutrients will diffuse through the nylon membrane to the encapsulated cells. The user can place the test strip back into its packaging, let the cells incubate, and check for light after an appropriate period of time, generally in the range of about 10 to about 30 minutes, depending on the chemical to be determined and the particular characteristics of the modified bioreporter organism.

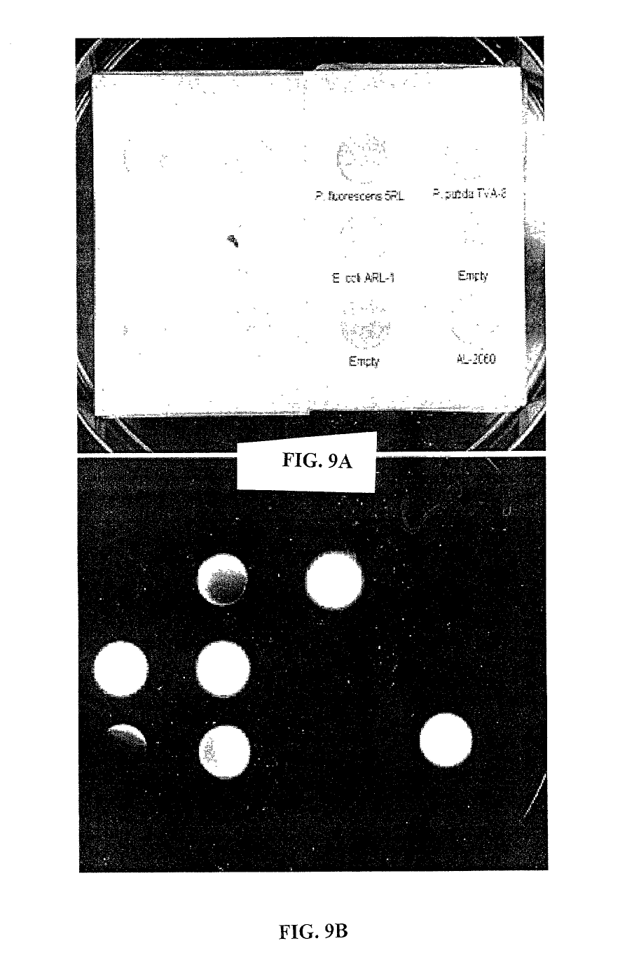

FIG. 9 shows the prototype test strip. In the upper panel, the test strip on the left side contains P. fluorescens 5RL induced by salicylate. The bioluminescence is readily observed in the lower panel. In the upper panel on the right side contains 3 bioreporters (P. fluorescens 5RL, P. putida TVA8, and E. coli ARL1) and the positive control (P. putida AL-2060). These cells were exposed to 1 ppm Hg2+, 10 ppm toluene, and 5 ppm naphthalene. In this particular case, the positive control and the naphthalene bioreporter were visible to the naked eye. The bioreporters for toluene and mercury were only detectable with a photomultiplier.

### 5.8 Example 8

### Bioluminescence Production by Immobilized Cells

Bioluminescence production in the proposed test strip was simulated using immobilized cells in a microtiter plate. This format was used so bioluminescence could be measured over time in response to simulated contaminated water. The procedure for this experiment was as follows:

1. A 500 ml culture of the cells was grown on mineral salts medium (MSM), trace elements, antibiotics, and 10 mM glucose.

2. The cells were harvested by centrifugation at 5,000 rpm for 30 minutes and washed 3 times with phosphate buffered saline (PBS).

3. The cell pellets were resuspended in MSM, trace elements, 10 mM glucose and stored at 4° C. until needed.

4. Blotting paper was cut into small circles (˜2 cm), the same size of the wells of a 12-well microtiter plate.

5. Noble agar (1.7%) was made using MSM and autoclaved. Trace elements and 10 mM glucose was added after autoclaving.

6. Two ml of the cells was pipetted into a 2 ml centrifuge tube and centrifuged at 10,000 rpm for 10 minutes. The pellet was resuspended in an additional 2 ml of cells and centrifuged as before. Approximate cell concentrations were:

E. coli ARL1 (Hg2+ detection)—7.2×1010

P. putida TVA8 (toluene detection)—3.92×1010

P. putida AL-2060 (positive control)—9.6×1010

P. fluorescens 5RL (naphthalene detection)—1.16×1011

7. The pellet was resuspended in 200 ul of MSM, trace elements, 10 mM glucose and 800 ul of agar was added. The mixture was kept at 45° C. in a water bath

8. The styrene mold was pressed onto a layer of silicone plastic (Sylgard 184 Silicone Elastomer Kit, DOW Coming, Midland, Mich.) to create a seal around the well and the noble agar/cell mixture was pipetted into each well. The patches were prepared one strain at a time.

9. While the patches were solidifying, 300 μl of a mixture of toluene (105 ppm), naphthalene (6.6 ppm), and mercury (II) chloride (0.2 ppm) prepared in LB was added to the wells.

10. The styrene mold was removed and the patches were moved using forceps. The patches were placed on top of the saturated blotting paper.

11. The same procedure was repeated for all of the strains.

12. The plate was read on a Wallac Victor2 (Perkin-Elmer Wallac, Gaithersburg, Md.) set to read the plate 10 minute for 99 cycles.

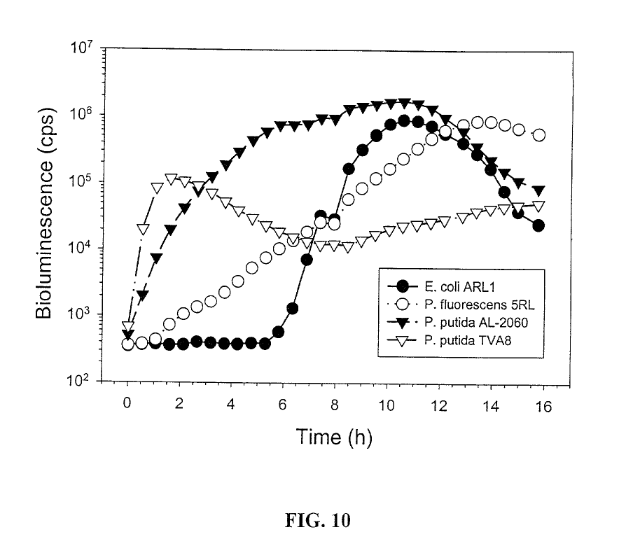

FIG. 10 shows bioluminescence production over time in the immobilized cells. Each strain tested produced bioluminescence in response to its specific analyte. One skilled in the art will recognize that improved response may be obtained by optimizing cell number and medium composition. Calibration curves for each strain may also be developed to allow quantification.

### 5.9 Example 9

### Alternate Test Strip Design

To extend the shelf life of the bioreporters on the test strip, the cells can be lyophilized before immobilization. The bioreporters can be rehydrated when the test strip is immersed in water. In this example, the test strip is the size of a credit card (84×54 mm) and has five layered parts as shown in FIG. 9, each described as follows:

1. The bottom layer is absorbent Whatman filter paper. The dimensions of this filter paper are 89×54 mm. The filter paper is prepared by steam sterilization for 20 minutes followed by a 10 minute drying cycle. The sterile filter paper is soaked in YEPG broth (yeast extract (0.2 g/L)—peptone (2.0 g/L)—glucose (1.0 g/L)—NH₄NO₃ (0.2 g/L)). After the filter is saturated, the excess broth is decanted, the filter paper frozen at −80° C., and lyophilized. The result is an absorbent filter paper impregnated with freeze-dried YEPG. This will serve as a nutrient source for the bioreporters when they are ‘rehydrated’ with a contaminated water sample.

2. The second layer is the styrene sheet. It acts as a semi-rigid support for the device as well as a mold for the encapsulated cells. Currently we are using styrene that is 0.5-1.0 mm thick. One-half inch circles are excised from the sheet (FIG. 9, top view). There is one circle for each bioreporter.

3. The third layer is lyophilized bioreporters. The current procedure is to grow the cells overnight in minimal salts medium (MSM) supplemented with trace elements and glucose. Cells are harvested by centrifugation at 5,000 rpm for 15 minutes. Cells are washed three times in either nitrogen-free MSM or phosphate-buffered saline. The cells are resuspended in 10% skim milk powder (Difco Laboratories, Detroit, Mich.).

4. The last layer is dialysis membrane to seal the lyophilized cells in place. The dialysis membrane also allows the diffusion of oxygen to the cells as well as target analytes.

The device is packaged in an air-tight, water tight material. Exemplary packaging may be either a heat-sealable foil or heat-sealable plastic (such as a “Seal-a-Meal” system).

### 5.10 Example 10

### Construction of merRo/pA-lux Bioreporter

A mercury bioreporter with the mercuric reductase gene (merA) fused to the merRo/p may be used to mitigate the toxic effects of high concentrations of mercury thus ensufiring a biolumninescent response. The merA gene was PCR amplified using primer sequences 3 and 4 (Table 3). The DNA template was pDG106 (Gambill and Summers, 1985). The amplified gene was cloned into the XbaI site in the merRo/p fragment. The merRo/pA will be excised from pCR2.1 with EcoRV and BamHI. The purified fragment will be cloned in Plasmid pFSP3 as described in section 5.1.

TABLE 3
Primers for Amplification of mer Components

| Primer | Sequence | Notes |
|---|---|---|
| 19145-NotI | 5′-GCGGCCGCttgaattggattggatagcgtaaccttacttccg | Incorporation of NotI |
| | SEQ ID NO:1 | Site at 5′-end of merRo |
| 19638-XbaI | 5′-AGATCTctaaggcatagctgacc | Opposite strand; |
| | SEQ ID NO:2 | Incorporation of XbaI site |
| 16289-XbaI | 5′-TCTAGAcacccggcgcagcaggaaagctgc | Incorporation of XbaI site |
| | SEQ ID NO:3 | |
| 17995-SPeI | 5′-ACTAGTaggaacgatggtatgagcactctcaaaatcacc | Opposite strand; |
| | SEQ ID NO:4 | Incorporation of SpeI site |

*Capital letters denote the addition of the restriction site

### 5.11 Example 11

### Bioreporter Characteristics

All genetically engineered strains were tested for sensitivity to the inducer compound (Hg²⁺) from 0.02 ppb to 10 ppm, dynamic range of bioluminiscent response, linearity of the response curve, response time vs. Hg²⁺. These assays are used in a microtiter plate format and light detected using Wallace MicroBeta Instrument. Simultaneously, a replicate set of plates will be observed with night vision goggles. Physical observation data will be correlated with the Wallace, Hg²⁺ concentration, and cell number.

### 5.12 Example 12

### Immobilization of Cells to a Handheld Device

Alginate has been used successfully for encapsulation of cells without adverse affects on viability. Long-term viability (weeks to months) is possible as long as the alginate-encased cells remain moist. Latex copolymers have been reported to be useful for immobilizing E. coli and maintaining viability (Lyngberg et al., 1999a; Lyngberg, et al., 1999b).

Acrylic vinyl acetate copolymer latex (Lyngberg, et al, 1999A, 1999B) has been reported as a useful immobilization medium. Lyngberg, et al. (1999A) immobilized E. coli in Rovace SF091 (Rohm and Haas, Philadelphia, Pa.). The authors demonstrated immobilization in highly uniform, reproducible patches and maintained viable but non-growing E. coli cells over a 15-day period. Lyngberg et al. (1999B) immobilized E. coli HB101 with the Selifonova et al. (1993) plasmids in latex patches. The patches detected Hg⁺ over a wide range of concentrations (0.1 to 10,000 nM). Response times varied, up to 5 hr depending on the Hg⁺ concentration in the sample (Lyngberg,1999A, 1999B).

### 5.13 Example 13

### Immobilization of Bioreporter Microorganisms

In an example using strain P. fluorescens 5RL, a response time of less than 60 minutes using salicylate as the inducer was observed (FIG. 10). Filter strips were immersed in a broth culture of P. fluorescens 5RL. Strips were removed and excess fluid was drained. These filter strips with cells were placed on a second set of strips impregnated with salicylic acid (10 and 100 ppm). The bacteria immobilized in this type of filter paper began to emit light in less than 30 minutes (observable with a liquid light pipe and photomultiplier) and light was visible to the naked eye in less than 60 minutes. This experiment demonstrated that bioluminescence can be observed with the naked eye.

Briefly, a 100 μl sample of an uninduced culture of Pseudomonas fluorescens 5RL was placed in a sterile petri dish containing LB broth (10 ml) supplemented with tetracycline (14 μg/mI). The culture was placed in a 30° C. incubator for 48 hr. Strips of cellulose-based filter paper (Fisher brand P8) 10 cm×2 cm were cut and sterilized by autoclaving at 255° C. for 25 min. The sterile strips of filter paper were immersed in the P. fluorescens 5RL for 15 min at which time the strips were removed and excess fluids allowed to drain.

Separately, 2 sterile cellulose filter disks were dipped in LB broth supplemented with tetracycline (14 μg/ml and salicylate at two different concentrations (10 ppm and 100 ppm). The disks were removed, excess fluid drained, and the disks placed in a sterile petri dish. The P. fluorescens 5 RL impregnated filter strips were placed on top of the filter disks and incubated at room temperature until light was visible to the naked eye. The immobilized bacteria emitted light in less than 30 min (detectable with an Oriel photomultiplier Model 7070) and emitted enough light to be visible to the naked eye in less than 60 min.

### 5.14 Constructs for Detection of Environmental Toxins

### 5.14.1 Example 15

### Bioluminescent Bioreporters for Naked Eye Visualization

In addition to the system developed for detection of mercury compounds, it is contemplated that the four additional chemical bioluminescent bioreporters can be immobilized on a filter strip matrix and detected visually without need for optical detection instruments. These reporters for toluene, naphthalene, and phenol are partially characterized (Table 4). One control will also be employed. Pseudomonas putida 2440 (pUTK2) contains the lux cassette behind a constitutive promoter; i.e. bioluminescence is always turned on. This strain will be the positive control to indicate the integrity of the filter test strip.

TABLE 4
Bioluminescent bioreporters for filter strip development.

| | | Lower | | |
|---|---|---|---|---|
| | | Detection | Response | |
| Bioreporter | Target | Limits | Time | Reference |
| P. fluorescens 5RL | Naphthalene | ˜50 ppb | ˜1 hour | King |
| (pUTK21) | (Tc^{r}) | | | et al., |
| | | | | 1990 |
| P. putida TVA8 | Toluene (Km^{r}) | 50 ppb | 25 ppm | Applegate |
| | | | | et al., |
| | | | | 1998 |
| Escherichia coli | Mercury (II) | ˜1 ppb | <1 | |
| (pOS14) | (Km^{r}) | | | |
| Acinetobacter | Phenol | TBD | TBD | |
| calcoaceticus DF42 | (Km^{r}) | | | |
| P. putida 2440 | Constitutive | — | — | |
| (pUTK2) | (Tc^{r}) | | | |

### 5.15 Example 15

### Cells Required for Generation of Sufficient Bioluminescence for Direct Visualization

Observable bioluminescence is expected be equivalent among the strains present on the test strip. Each strain will be tested at an environmentally appropriate concentration and bioluminescent output will be measured as a function of cell number. These tests will be performed in liquid and on a solid support matrix such as cellulose or a nylon filter strip. The number of cells applied to the filter strip will be adjusted to give equivalent levels of light sufficient to be viewed by the human eye.

### 5.16 Example 16

### Functional Concentration Range of Immobilized Cells for Each Contaminant

All bioreporters have been tested in liquid culture. The functional concentration range of each contaminant may be different when cells are immobilized to a solid support. The effective bioluminescent response for each contaminant over a selected range in liquid and when immobilized on the solid support.

### 5.17 Example 17

### Sensitivity of Immobilized Cells to Each Contaminant

Filter strips with immobilized cells will be tested for sensitivity to the inducer compounds (Table 1). In addition, the dynamic range of the bioluminescent response, linearity of the response curve, and response time will be determined using laboratory light detection equipment, direct observation, and night vision equipment.

### 6.0 SEQUENCE LISTING

SEQ ID NO: 1

5′-GCGGCCGCttgattggattggagcgyaaccttacttccg

SEQ ID NO:2

5′-AGATCT ctaaggcatagctgacc

SEQ ID NO:3

5′-TCTAGAcacccggcgcagcaggaaagctgc

SEQ ID NO:4

5′-ACT AGT aggaacgatggtatgagcactctcaaaatcacc

### 7.0 REFERENCES

Agency for Toxic Substances and Disease Registry Division of Toxicology, Public Health Statement, 1990.

Applegate, B. M., S. R. Kehrmeyer, and G. S. Sayler. 1998. A chromosomally based tod-luxCDABE whole-cell reporter for benzene, toluene, ethylbenzene, and xylene (BTEX) sensing. Appl. Environ. Microbiol. 64, 2730-2735.

Burlage, R. S., L. A. Bemis, A. C. Layton, G. S. Sayler, and F. Larimer. 1990. Comparative genetic organization of incompatibility group P degradative plasmids. J. Bacteriol. 172:6818-6825.

Contag, C. H. et al. (2000) Use of reporter genes for optical measurements of neoplastic disease in vivo. Neoplasia 2 (1-2), 41-52

De Lorenzo, Fernandez, Herrero, Jakubik, and Timmis, “Engineering of alkyl- and haloaromatic-responsive gene expression with mini-transposons containing regulated promoters of biodegradative pathways of Pseudomonas,” Gene, 130:41-46, 1993.

Folsom, B. R., P. J. Chapman, and P. H. Pritchard. 1990. Phenol and trichloroethylene degradation by Pseudomonas cepacia G4: Kinetics and interactions between substrates. Appl. Environ. Microbiol. 56:1279-1285.

Gibson, D. T., J. R. Koch, and R. E. Kallio. 1968. Biochemistry. 7:2653-2662.

Hay, A. G., Applegate, B. M., Bright, N. G., and Sayler, G. S. “A bioluminescent whole-cell reporter for the detection of 2,4-dichlorophenoxyacetic acid,” Appl. Envir. Microbiol., 66, 4589-4594, 2000.

Hermens, J. et al. (1985) Quantitative structure-activity relationships and mixture toxicity of organic chemicals in Photobacterium phosphoreum: the Microtox test. Ecotoxicol. Environ. Saf 9, 17-25

King, J. M. H., P. M. DiGrazia, B. Applegate, R. Burlage, J. Sanseverino, P. Dunbar, F. Larimer, and G. S. Sayler. 1990. Rapid, Sensitive Bioluminescent Reporter Technology for Naphthalene Exposure and biodegradation. Science. 249:778-781, 1991.

Layton, Muccini, Ghosh and Sayler, “Construction of a Bioluminescent Reporter Strain To Detect Polychlorinated Biphenyls,” Appl. Envir. Microbiol., 64:5023-5026, 1998.

Legler, J. et al. (1999) Development of a stably transfected estrogen receptor-mediated luciferase reporter gene assay in the human T47D breast cancer cell line. Toxicol. Sci. 48(1), 55-66.

Lyngberg, Thiagarajan, Sternke, Schottel, Scriven, and Flickinger, “A patch coating method for preparing biocatalytic films of Escherichia coli ” Biotechnol. Bioeng. 62:44-55, 1999a.

Lyngberg, Sternke, Schotte, and Flickinger, “A single use luciferase-based mercury biosensor using Escherichia coil HB 101 immobilized on a latex copolymer film” J. Industrial Microbiology and Biotechnol., 23 :668-675, 1999b.

Meighen, E. A. (1994) Genetics of bacterial bioluminescence. Annu. Rev. Genet. 28, 117-139

Misteli, T. and Spector, D. L. (1997) Application of the green fluorescent protein in cell biology and biotechnology. Nat. Biotechnol. 15, 961-964.

Petanen, T., M. Virta, M. Karp, and M. Romantschuk. 2001. Contruction and use of broad host range mercury and arsenite sensor plasmids in the soil bacterium Pseudomonas fluorescens OS8. Microbial Ecol. (41):360-369.

Rider, T. et al. (1999) In NASA/NCI Biomedical Imaging Symposium.

Sanseverino, J., B. Applegate, J. M. H. King, and G. S. Sayler, 1993, “The Naphthalene Degradative Pathway of NAH7 and NAH7-Like Plasmids Mineralizes Phenanthrene and Anthracene,” Applied and Environmental Microbiology, Vol. 59 pp. 1931-1937.

Sattler, I. et al. (1995) Cloning, sequencing, and expression of the uroporphyrinogen-III methyltransferase cobA gene of Propionibacterium freudenreichii (shermanii). J. Bacteriol. 177, 1564-1569

Selifonova, Burlage, and Barkay, “Bioluminescent Sensors for Detection of Bioavailable Hg(II) in the Environment,” Appl. Environ. Microbiol., 59:3083-3090, 1993.

Summers, A. O. 1986. Organization, expression, and evolution of genes for mercury resistance. Ann. Rev. Microbiol. 40:607-634.

Virta, M., J. Lampinen, and M. Karp. 1995. A luminescence-based mercury biosensor. Anal. Chem. 67:667-669.

What is claimed is:

1. A device for detecting a selected analyte, comprising:
a stably transformed bacterium containing a promoterless lux gene cassette having a regulatory element for a selected analyte inserted in front of the lux gene cassette;
a support matrix onto which the bacterium is attached; and
an encapsulating material to contain said bacterium attached to the support matrix
wherein the encapsulated bacterium emits visibly detectable light in the presence of the selected analyte.

2. The device of claim 1 wherein the lux gene cassette comprises merRo/p-lux.

3. The device of claim 2 further comprising a merRo/pA-lux gene cassette incorporated into the transformed bacterium.

4. The device of claim 1 wherein the analyte is naphthalene, toluene, ethylbenzene, 2,4-dichlorophenoxyacetic acid, β-phenyl ethylamine, phenol or bipheny.

5. The device of claim 1 wherein the analyte is mercury.

6. The device of claim 1 wherein the regulatory element comprises a mer regulatory element.

7. The device of claim 4 wherein the regulatory element further comprises a mer operator.

8. The device of claim 1 wherein the bacterium is P. fluorescens.

9. The device of claim 6 wherein the P. fluorescens is P. fluorescens 5R.

10. An apparatus comprising the device of claim 1.

11. The apparatus of claim 10 comprising a holder for the support matrix onto which the bacterium is immobilized.

12. The apparatus of claim 11 adapted to hand-carrying.

13. A genetically modified bacterium responsive to divalent mercury, said bacterium containing a merRo/p-lux gene stably integrated into the bacterial chromosome wherein said bacterium produces a bioluminescent protein in the presence of divalent mercury.

14. The genetically modified bacterium of claim 13 that is encapsulated.

15. The genetically modified bacterium of claim 14 that is encapsulated in a matrix selected from the group consisting of alginate, carrageenan, acrylic vinyl acetate copolymer, latex, polyvinyl chloride polymer, sol-gels, agar, agarose, micromachined nanoporous membranes, polydimethylsiloxane (PDMS), polyacrylamide, polyurethane/polycarbamyl sulfonate and polyvinyl alcohol.

16. The encapsulated genetically modified bacterium of claim 14 that is attached to a support matrix.

17. The encapsulated genetically modified bacterium of claim 16 wherein the support matrix is cellulose, glass, colloidal noble metal, plastic, laminin or resin.

18. A cellulose support comprising the genetically modified bacterium of claim 13.

19. A kit for detecting mercury II ion comprising the genetically modified bacterium of claim 13 adhered to an immobilization support and instructions for use in detecting mercury ion.

20. The kit of claim 19 further comprising a second genetically modified bacterium harboring a stably integrated merRo/pA-lux gene.

21. The kit of claim 19 or claim 20 further comprising a direct visual assistance device.

22. The kit of claim 21 wherein the direct visual assistance device is a light-tight box or night vision goggles.

23. The kit of claim 19 or 20 wherein the genetically modified bacterium is P. putida 2440, P. fluorescens 5R, P. putida F1, Escherichia coli, Vibrio fischerii, Vibrio harveyi, or Bacillus subtilis.

24. The kit of claim 23 wherein the bacterium is P. fluorescens 5R.

25. A method for detecting mercury comprising
contacting a sample suspected of containing mercury II ion with a bioreporter bacterium genetically modified to contain a merRo/pA-lux gene; and
detecting the presence of the mercury ion when a visibly detectable luminescence is produced.

26. The method of claim 25 wherein the bioreporter bacterium is E. coli ARL1, ARL2 or ARL3.

27. The method of claim 25 wherein the bioreporter is encapsulated or immobilized.
